SEQUENCE LISTING (42 sequences)

SEQ ID NO: 1 - protein, 401 aa, synthetic construct
GVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPY

SEQ ID NO: 2 - protein, 401 aa, synthetic construct
GVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPY

SEQ ID NO: 3 - protein, 439 aa, synthetic construct
GVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPTCSQDEFRCHDGKCISRQFVCDSDRDCLDGSDEASCPGY

SEQ ID NO: 4 - protein, 439 aa, synthetic construct
GVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPTCSQDEFRCHDGKCISRQFVCDSDRDCLDGSDEASCPGY

SEQ ID NO: 5 - protein, 389 aa, synthetic construct
GVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVTCSQDEFRCHDGKCISRQFVCDSDRDCLDGSDEASCPGY

SEQ ID NO: 6 - protein, 831 aa, synthetic construct
TAGCCAGGGACGCCAACACCCGGTACACCCAGACCTGGCACACCGACGCCCGGAACACCCAGCCCCGGAACGCCCACACCAGGGACGCCAACACCCGGTACACCCAGACCTGGCACACCGACGCCCGGAACACCCAGCCCCGGAACGCCCACACCAGGGACGCCAACACCCGGTACACCCAGACCTGGCACACCGACGCCCGGAACACCCAGCCCCGGAACGCCCACACCAGGGACGCCAACACCCGGTACACCCAGACCTGGCACACCGACGCCCGGAACACCCAGCCCCGGAACGCCCACACCAGGGACGCCAACACCCGGTACACCCAGACCTGGCACACCGACGCCCGGAACACCCAGCCCCGGAACGCCCACACCAGGGACGCCAACACCCGGTACACCCAGACCTGGCACACCGACGCCCGGAACACCCAGCCCCGGAACGCCCACACCAGGGACGCCAACACCCGGTACACCCAGACCTGGCACACCGACGCCCGGAACACCCAGCCCCGGAACGCCCACACCAGGGACGCCAACACCCGGTACACCCAGACCTGGCACACCGACGCCCGGAACACCCAGCCCCGGAACGCCCACACCAGGGACGCCAACACCCGGTACACCCAGACCTGGCACACCGACGCCCGGAACACCCAGCCCCGGAACGCCCACACCAGGGACGCCAACACCCGGTACACCCAGACCTGGCACACCGACGCCCGGAACACCCAGCCCCGGAACGCCCACACCAGGGACGCCAACACCCGGTACACCCAGACCTGGCACANCGACGCCCGGAACACCCAGCCCCGGAACGCCCACACCA

SEQ ID NO: 7 - protein, 804 aa, synthetic construct
TAGCCCGGCACGCCGACACCAGGAACACCAACGCCCGGTACGCCCACACCTGGGACACCTACGCCCGGAACACCCACGCCCGGCACGCCGACACCAGGAACACCAACGCCCGGTACGCCCACACCTGGGACACCTACGCCCGGAACACCCACGCCCGGCACGCCGACACCAGGAACACCAACGCCCGGTACGCCCACACCTGGGACACCTACGCCCGGAACACCCACGCCCGGCACGCCGACACCAGGAACACCAACGCCCGGTACGCCCACACCTGGGACACCTACGCCCGGAACACCCACGCCCGGCACGCCGACACCAGGAACACCAACGCCCGGTACGCCCACACCTGGGACACCTACGCCCGGAACACCCACGCCCGGCACGCCGACACCAGGAACACCAACGCCCGGTACGCCCACACCTGGGACACCTACGCCCGGAACACCCACGCCCGGCACGCCGACACCAGGAACACCAACGCCCGGTACGCCCACACCTGGGACACCTACGCCCGGAACACCCACGCCCGGCACGCCGACACCAGGAACACCAACGCCCGGTACGCCCACACCTGGGACACCTACGCCCGGAACACCCACGCCCGGCACGCCGACACCAGGAACACCAACGCCCGGTACGCCCACACCTGGGACACCTACGCCCGGAACACCCACGCCCGGCACGCCGACACCAGGAACACCAACGCCCGGTACGCCCACACCTGGGACACCTACGCCCGGAACACCCACGCCCGGCACGCCGACACCANGAACACCAACGCCCGGTACGCCCACACCTGGG

SEQ ID NO: 8 - protein, 720 aa, synthetic construct
TAGCCCGGGCAGCTGGCTTCATCGCTGCCATCCAGACAATCGCGATCGCTATCGCAAACGAACTGGCGGCTGATGCATTTGCCATCATGACAGCGGAACTCATCTTGGCTGCACGTGCCAGGGACGCCAACACCCGGTACACCCAGACCTGGCACACCGACGCCCGGAACACCCAGCCCCGGAACGCCCACACCAGGGACGCCAACACCCGGTACACCCAGACCTGGCACACCGACGCCCGGAACACCCAGCCCCGGAACGCCCACACCAGGGACGCCAACACCCGGTACACCCAGACCTGGCACACCGACGCCCGGAACACCCAGCCCCGGAACGCCCACACCAGGGACGCCAACACCCGGTACACCCAGACCTGGCACACCGACGCCCGGAACACCCAGCCCCGGAACGCCCACACCAGGGACGCCAACACCCGGTACACCCAGACCTGGCACACCGACGCCCGGAACACCCAGCCCCGGAACGCCCACACCAGGGACGCCAACACCCGGTACACCCAGACCTGGCACACCGACGCCCGGAACACCCAGCCCCGGAACGCCCACACCANGGACGCCAACACCCGGTACACCCAGACCTGGCACACCGACGCCCGGAACACCCAGCCCCGGAACGCCCACACCNNGNACGCCAACACCCGGTACACCCAGACCTGGCACACCGACGCCCGGAACACCCAGCCCCGGAACGCCCACACCN

SEQ ID NO: 9 - protein, 809 aa, synthetic construct
TAGCCCGGGCAGCTGGCTTCATCGCTGCCATCCAGACAATCGCGATCGCTATCGCAAACGAACTGGCGGCTGATGCATTTGCCATCATGACAGCGGAACTCATCTTGGCTGCACGTGCCCGGCACGCCGACACCAGGAACACCAACGCCCGGTACGCCCACACCTGGGACACCTACGCCCGGAACACCCACGCCCGGCACGCCGACACCAGGAACACCAACGCCCGGTACGCCCACACCTGGGACACCTACGCCCGGAACACCCACGCCCGGCACGCCGACACCAGGAACACCAACGCCCGGTACGCCCACACCTGGGACACCTACGCCCGGAACACCCACGCCCGGCACGCCGACACCAGGAACACCAACGCCCGGTACGCCCACACCTGGGACACCTACGCCCGGAACACCCACGCCCGGCACGCCGACACCAGGAACACCAACGCCCGGTACGCCCACACCTGGGACACCTACGCCCGGAACACCCACGCCCGGCACGCCGACACCAGGAACACCAACGCCCGGTACGCCCACACCTGGGACACCTACGCCCGGAACACCCACGCCCGGCACGCCGACACCAGGAACACCAACGCCCGGTACGCCCACACCTGGGACACCTACGCCCGGAACACCCACGCCCGGCACGCCGACACCANGAACACCAACGCCCGGTACGCCCACACCTGGGACACCTACGCCCGGAACACCCACGCCCGGCACGCCGACACCANGAACACCAACGCCCGGTACGCCCACACCTGGGACACCTACGCCCGGAACACCCACGCCCGGCACGCCGACACC

SEQ ID NO: 10 - protein, 765 aa, synthetic construct
TAGCCCGGGCAGCTGGCTTCATCGCTGCCATCCAGACAATCGCGATCGCTATCGCAAACGAACTGGCGGCTGATGCATTTGCCATCATGACAGCGGAACTCATCTTGGCTGCACGTGCCAGGAACACCCACACCTGGAACGCCTGCTCCTGGAACACCCACGCCTGGCACTCCGGCGCCAGGAACACCCACACCTGGAACGCCTGCTCCTGGAACACCCACGCCTGGCACTCCGGCGCCAGGAACACCCACACCTGGAACGCCTGCTCCTGGAACACCCACGCCTGGCACTCCGGCGCCAGGAACACCCACACCTGGAACGCCTGCTCCTGGAACACCCACGCCTGGCACTCCGGCGCCAGGAACACCCACACCTGGAACGCCTGCTCCTGGAACACCCACGCCTGGCACTCCGGCGCCAGGAACACCCACACCTGGAACGCCTGCTCCTGGAACACCCACGCCTGGCACTCCGGCGCCAGGAACACCCACACCTGGAACGCCTGCTCCTGGAACACCCACGCCTGGCACTCCGGCGCCAGGAACACCCACACCTGGAACGCCTGCTCCTGGAACACCCACGCCTGGCACTCCGGCGCCAGGAACACCCACACCTGGAACGCCTGCTCCTGGAACACCCACGCCTGGCACTCCGGCGCCAGGAACACCCACACCTGGAACGCCTGCTCCTGGAACACCCACGCCTGGCACTCCGGCGCCAGGAACACCCACACCTGGNACGCCTGCTCCTGGAACACCCACGCCT

SEQ ID NO: 11 - protein, 400 aa, synthetic construct
GVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPV

SEQ ID NO: 12 - protein, 400 aa, synthetic construct
GVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVP

SEQ ID NO: 13 - protein, 400 aa, synthetic construct
GVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVP

SEQ ID NO: 14 - protein, 39 aa, synthetic construct
TCSQDEFRCHDGKCISRQFVCDSDRDCLDGSDEASCPGY

SEQ ID NO: 15 - protein, 443 aa, synthetic construct
SKGPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPTCSQDEFRCHDGKCISRQFVCDSDRDCLDGSDEASCPGY

SEQ ID NO: 16 - protein, 443 aa, synthetic construct
SKGPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPTCSQDEFRCHDGKCISRQFVCDSDRDCLDGSDEASCPGY

SEQ ID NO: 17 - protein, 393 aa, synthetic construct
SKGPGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVTCSQDEFRCHDGKCISRQFVCDSDRDCLDGSDEASCPGY

SEQ ID NO: 18 - protein, 444 aa, synthetic construct
MSKGPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPTCSQDEFRCHDGKCISRQFVCDSDRDCLDGSDEASCPGY

SEQ ID NO: 19 - protein, 444 aa, synthetic construct
MSKGPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPTCSQDEFRCHDGKCISRQFVCDSDRDCLDGSDEASCPGY

SEQ ID NO: 20 - protein, 394 aa, synthetic construct
MSKGPGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVTCSQDEFRCHDGKCISRQFVCDSDRDCLDGSDEASCPGY

SEQ ID NO: 21 - protein, 5 aa, synthetic construct
  REPEAT 1..5: note = n=1-20
EAAAK

SEQ ID NO: 22 - protein, 5 aa, synthetic construct
  REPEAT 1..5: note = n=1-30
GGGGS

SEQ ID NO: 23 - protein, 4 aa, synthetic construct
  REPEAT 1..4: note = n=1-50
SGGG

SEQ ID NO: 24 - protein, 4 aa, synthetic construct
  REPEAT 1..4: note = n=1-20
GGGS

SEQ ID NO: 25 - protein, 18 aa, synthetic construct
KESGSVSSEQLAQFRSLD

SEQ ID NO: 26 - protein, 14 aa, synthetic construct
EGKSSGSGSESKST

SEQ ID NO: 27 - skipped

SEQ ID NO: 28 - protein, 12 aa, synthetic construct
GKSSGSGSESKS

SEQ ID NO: 29 - protein, 14 aa, synthetic construct
GSTSGSGKSSEGKG

SEQ ID NO: 30 - protein, 18 aa, synthetic construct
GSTSGSGKSSEGSGSTKG

SEQ ID NO: 31 - protein, 18 aa, synthetic construct
GSTSGSGKPGSGEGSTKG

SEQ ID NO: 32 - protein, 14 aa, synthetic construct
EGKSSGSGSESKEF

SEQ ID NO: 33 - protein, 5 aa, synthetic construct
SRSSG

SEQ ID NO: 34 - protein, 5 aa, synthetic construct
SGSSC

SEQ ID NO: 35 - protein, 18 aa, synthetic construct
EGRGSLLTCGDVEENPGP

SEQ ID NO: 36 - protein, 19 aa, synthetic construct
ATNFSLLKQAGDVEENPGP

SEQ ID NO: 37 - protein, 20 aa, synthetic construct
QCTNYALLKLAGDVESNPGP

SEQ ID NO: 38 - protein, 22 aa, synthetic construct
VKQTLNFDLLKLAGDVESNPGP

SEQ ID NO: 39 - protein, 201 aa, synthetic construct
GVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPY

SEQ ID NO: 40 - protein, 600 aa, synthetic construct
GGTGTAGGCGTCCCGGGAGTAGGCGTCCCGGGGGCCGGAGTGCCGGGCGTCGGGGTCCCCGGAGTGGGCGTGCCCGGGGTAGGGGTTCCCGGTGTAGGAGTCCCGGGCGCTGGTGTACCGGGGGTCGGAGTACCGGGCGTGGGTGTGCCCGGTGTTGGTGTCCCCGGTGTGGGAGTGCCGGGCGCTGGCGTACCTGGTGTGGGAGTACCTGGGGTCGGCGTTCCGGGCGTTGGAGTCCCCGGTGTGGGCGTTCCTGGTGCGGGCGTTCCGGGAGTCGGAGTGCCTGGGGTGGGAGTGCCGGGGGTCGGTGTACCCGGTGTGGGTGTCCCCGGAGCGGGGGTACCGGGCGTTGGCGTACCCGGCGTCGGGGTTCCGGGCGTTGGCGTACCTGGAGTCGGCGTGCCCGGCGCGGGCGTCCCGGGCGTCGGCGTACCGGGAGTAGGTGTTCCCGGGGTCGGAGTCCCGGGCGTAGGTGTCCCGGGCGCGGGCGTCCCCGGGGTCGGAGTGCCTGGAGTGGGCGTCCCCGGAGTGGGCGTGCCCGGAGTAGGCGTTCCGGGGGCGGGAGTACCCGGCGTGGGAGTACCGGGCGTAGGGGTGCCA

SEQ ID NO: 41 - protein, 200 aa, synthetic construct
GVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVP

SEQ ID NO: 42 - protein, 350 aa, synthetic construct
GVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPV

(19) United States
(12) Patent Application Publication
(10) Pub. No.: US 2026/0357295 A1
(43) Pub. Date: Nov. 12, 2026

(54) VIRAL TRANSDUCTION REAGENTS AND METHODS OF USE

(71) Applicant: Donaldson Company, Inc., Bloomington, MN (US)

(72) Inventors: Nicole Votaw, Durham, NC (US); Alyssa Wheeler, Durham, NC (US)

(21) Appl. No.: 19/258,400

(22) Filed: Jul. 2, 2025

Related U.S. Application Data
(60) Provisional application No. 63/667,372, filed on Jul. 3, 2024.

(51) Int. Cl. C12N 15/86; C12Q 1/70

(52) U.S. Cl. CPC C12N 15/86; C12Q 1/70; C12N 2740/15043; C12N 2740/15051

(57) ABSTRACT

Compositions including fusion proteins, viruses, and a synthetic charged polymer are provided. The composition includes a fusion protein reagent, one or more viral particles, and synthetic charged polymer, where the fusion protein reagent includes an affinity domain and a polypeptide with phase behavior, and where a molar ratio of the synthetic charged polymer to the fusion protein reagent is 400:1 to 4:1. The compositions may be used to control viral transduction. The compositions may be used to improve viral transduction. Methods of using the compositions to control of viral transduction and improve viral transduction are described.

### CROSS-REFERENCE TO RELATED APPLICATIONS

This application claims the benefit of U.S. Provisional Application Ser. No. 63/667,372, filed on Jul. 3, 2024, the disclosure of which is incorporated by reference herein in its entirety.

### SEQUENCE LISTING

This application contains a Sequence Listing electronically submitted via EFS-Web to the United States Patent and Trademark Office as an XML file entitled “0444.000260US01.xml” having a size of 49,184 bytes and created on Jun. 30, 2025. The information contained in the Sequence Listing is incorporated by reference herein.

### SUMMARY

Provided herein are compositions including fusion proteins, viruses, and a synthetic charged polymer. Typically, the fusion proteins include the CR3 domain of the low density lipoprotein receptor (LDLR) and polypeptides with phase behavior. Such fusion proteins may cause blocking of viral transduction. The compositions described herein unexpectedly improve viral transduction. The compositions described herein unexpectedly allow for control of viral transduction, including up-regulation and down-regulation of viral transduction. The compositions of the present disclosure may improve viral transduction by preventing blocking caused by the fusion proteins. The present disclosure further provides methods of controlling viral transduction.

According to an embodiment, a composition includes a fusion protein reagent, one or more viral particles, and synthetic charged polymer, where the fusion protein reagent includes an affinity domain and a polypeptide with phase behavior, and where a molar ratio of the synthetic charged polymer to the fusion protein reagent is 400:1 to 4:1. The synthetic charged polymer may include a cationic polymer. The synthetic charged polymer may include polybrene. The fusion protein reagent is an ISOTAG® reagent. The composition may further include one or more cells.

According to an embodiment, a method of transducing one or more cells includes: contacting the one or more cells with a composition that includes a fusion protein reagent, one or more viral particles, and synthetic charged polymer, where the fusion protein reagent includes an affinity domain and a polypeptide with phase behavior, and where a molar ratio of the synthetic charged polymer to the fusion protein reagent is 400:1 to 4:1. The synthetic charged polymer may include polybrene. The fusion protein reagent may be an ISOTAG® reagent. The method may further include calculating the transduction efficiency, where the transduction efficiency is higher than one or more cells contacted with a comparable composition lacking a synthetic charged polymer.

According to an embodiment, a method of transducing one or more cells includes: contacting the one or more cells with a composition that includes: a fusion protein reagent that includes an affinity domain and a polypeptide with phase behavior; one or more viral particles; and a synthetic charged polymer, where the synthetic charged polymer is included in the composition in an amount that prevents 10% or more of blocking of cell transduction by the fusion protein reagent. The synthetic charged polymer may include polybrene. The fusion protein reagent may be an ISOTAG® reagent. The molar ratio of the synthetic charged polymer to the fusion protein reagent may be 400:1 to 4:1. The weight ratio of the synthetic charged polymer to the fusion protein reagent is from 25:1 to 1:25. The synthetic charged polymer may prevent 20% or more, 30% or more, 40% or more, 50% or more, 60% or more, 70% or more, or 80% or more of blocking of cell culture transduction by the fusion protein reagent. The synthetic charged polymer may prevent up to 90% of blocking of cell transduction by the fusion protein reagent.

According to an embodiment, a method of measuring a concentration of transducing units in a viral preparation includes: contacting one or more cells with one or more viral particles and a fusion protein reagent to form an intermediate composition, the fusion protein reagent that includes an affinity domain and a polypeptide with phase behavior, the intermediate composition that includes a synthetic charged polymer at a concentration of 10 μg/mL or greater, and the one or more viral particles that includes a genetic payload; incubating the cell for 12 hours or longer; detecting infection in the one or more cells; and calculating the concentration of transducing units based on the number of infected cells. The genetic payload may include a detectable marker. The detectable marker may include a fluorescent protein. The detecting infection may include flow cytometry, fluorescent microscopy, cell imaging, or a plaque assay. The method may further include quantifying the number of genomes in the one or more viral particles to provide a concentration of viral genomes. The quantifying may include quantitative real-time PCR (qRT-PCR). The method may further include quantifying the number of viral particles. The quantifying may include enzyme-linked immunosorbent assay (ELISA). The method may further include comparing the percent recovery of viral particles and the viral titer, where the percent recovery of viral particles may be within 10% of the transducing units.

According to an embodiment, a kit for transducing a cell includes: a fusion protein reagent that includes an affinity domain and a polypeptide with phase behavior; one or more viral particles; a synthetic charged polymer; and instructions for transducing the cell by contacting the cell with a composition that includes the fusion protein reagent, the one or more viral particles, and the synthetic charged polymer, where a molar ratio of the synthetic charged polymer to the fusion protein reagent is from 400:1 to 4:1. According to an embodiment, a kit for purifying a virus includes: an ISOTAG® reagent; a binding buffer; a wash buffer; an elution buffer; a formulation buffer; a synthetic charged polymer; and instructions for purifying the virus by using the ISOTAG® reagent, the binding buffer, the wash buffer, the elution buffer, and the synthetic charged polymer. The synthetic charged polymer may include polybrene. The formulation buffer or the elution buffer may include the synthetic charged polymer at a concentration of 10 μg/mL or more. The instructions may include instructions to add the synthetic charged polymer to the ISOTAG® reagent at a weight ratio of the synthetic charged polymer to viral particles of 25:1 to 1:25. The instructions may include instructions to add the synthetic charged polymer to the ISOTAG® reagent at a molar ratio of 400:1 to 4:1.

According to an embodiment, a method of controlling viral transduction of a cell includes: contacting the cell with a first composition that includes one or more viral particles and a fusion protein reagent, causing down-regulation of viral transduction of the cell; and contacting the cell with a second composition that includes the one or more viral particles and a synthetic charged polymer, causing up-regulation of viral transduction of the cell. The fusion protein reagent may include a fusion protein with an affinity domain and a polypeptide with phase behavior. The cell may be a virus-producing cell capable of producing the one or more viral particles. The method may further include causing the cells to produce the one or more viral particles prior to the contacting of the cell with the first composition.

According to an embodiment, a method of controlling transduction of a virus-producing cell includes contacting the virus-producing cell with a fusion protein. According to an embodiment, a method of producing a virus includes contacting the cell with one or more reagents encoding one or more viral components and contacting the cell with a fusion protein. The fusion protein may include an ISOTAG® reagent. The synthetic charged polymer may include polybrene. The molar ratio of the synthetic charged polymer to the fusion protein reagent may be 400:1 to 4:1. The synthetic charged polymer may prevent or reverse 20% or more, 30% or more, 40% or more, 50% or more, 60% or more, 70% or more, or 80% or more of blocking of cell culture transduction by the fusion protein reagent. The synthetic charged polymer may prevent or reverse up to 90% of blocking of cell transduction by the fusion protein reagent.

### BRIEF DESCRIPTION OF DRAWINGS

FIG. 1 is a graph showing the transduction efficiency of cells treated with different concentrations of a fusion protein of the present disclosure (0 micromolar, 0.1 micromolar, 0.5 micromolar, or 1 micromolar). Cells were incubated in media lacking adjuvant or with adjuvant added at the time of transduction.

FIG. 2 is a graph showing the transduction efficiency of cells treated with different concentrations of a fusion protein of the present disclosure (0 micromolar, 0.1 micromolar, 0.5 micromolar, 1 micromolar, 2 micromolar, or 5 micromolar). Cells were incubated in media lacking adjuvant or with adjuvant added at the time of transduction.

FIG. 3 is a graph showing the cell viability of cells treated with different concentrations of a fusion protein of the present disclosure (0 micromolar, 0.1 micromolar, 0.5 micromolar, 1 micromolar, or 5 micromolar). Cells were incubated with the fusion protein for a total of 10 days with samples taken on days 1, 3, 6, and 10.

### DEFINITIONS

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “about” or “approximately” when preceding a numerical value indicates the value plus or minus a range of 10%. For example, “about 100” encompasses 90 and 110.

The term “substantially” as used herein has the same meaning as “nearly completely,” and can be understood to modify the term that follows by at least about 90%, at least about 95%, or at least about 98%. The term “not substantially” as used here has the same meaning as “not significantly,” and can be understood to have the inverse meaning of “substantially,” i.e., modifying the term that follows by not more than 10%, not more than 5%, or not more than 2%.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features described herein can be used in any combination.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, particular embodiments may be described in isolation for clarity. Thus, unless otherwise expressly specified that the features of a particular embodiment are incompatible with the features of another embodiment, the particular features, configurations, compositions, or characteristics may be combined in any suitable manner in one or more embodiments. Thus, features described in the context of one embodiment may be combined with features described in the context of a different embodiment except where the features are necessarily mutually exclusive.

As used herein, the term “fragment” as it refers to a protein or polypeptide includes a truncated form of the protein or polypeptide. For example, a fragment of LDLR may include about 1%, about 2%, about 3%, about 4% about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, or about 99% of the amino acids of full-length LDLR. A fragment of the CR3 domain of LDLR may contain about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, or about 99% of the amino acids of the full-length CR3 domain of LDLR. The full-length CR3 domain of LDLR has the amino acid sequence of SEQ ID NO: 14. In embodiments, the fragment of the CR3 domain of LDLR includes at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, or at least 38 amino acids of the CR3 domain of LDLR.

As used herein, the terms “peptide,” “polypeptide,” and “protein” are used interchangeably, and refer to a compound including amino acid residues covalently linked by peptide bonds. A protein must contain at least two amino acids, but no limitation is placed on the maximum number of amino acids that can include a protein's sequence. The term “peptide” may refer to a short chain of amino acids including, for example, natural peptides, recombinant peptides, synthetic peptides, or a combination thereof. Proteins and peptides may include, for example, biologically active fragments, substantially homologous polypeptides, oligopeptides, homodimers, heterodimers, variants of polypeptides, modified polypeptides, derivatives, analogs, and fusion proteins, among others.

The term “modification” as it refers to a polypeptide refers to mutation, deletion, or addition of one amino acid of the polypeptide. In some embodiments, the polypeptide is the CR3 domain of the LDLR. The location of a modification within the CR3 domain of the LDLR can be determined based on aligning the sequence of the polypeptide to SEQ ID NO: 14 (the amino acid sequence of the CR3 domain of the LDLR).

A “polynucleotide” is a sequence of nucleotide bases, and may be an RNA, a DNA or a DNA-RNA hybrid sequence (including both naturally occurring and non-naturally occurring nucleotides). In some embodiments, a polynucleotide is either a single or double stranded DNA sequence.

The term “percent identity” in the context of two or more nucleic acid or polypeptide sequences, refers to two or more sequences or subsequences that have a specified percentage of nucleotides or amino acid residues that are the same, when compared. Unless otherwise indicated, percent identity is determined using the EMBL's European Bioinformatics Institute (EMBL-EBI) tool EMBOSS Needle, which is available at ebi.ac.uk/Tools/psa/emboss_needle/. The following default parameters may be used for EMBOSS Needle Pairwise Alignment: Matrix=BLOSUM62; Gap Open=10; Gap Extension=0.5; End Gap Penalty=false; End Gap Open=10; End Gap Extend=0.5. In embodiments, the percent identity is calculated over the entire length of the compared sequences. In some embodiments, the sequence identity is calculated over a fragment of each compared sequence of about 10 amino acids, about 15 amino acids, about 20 amino acids, about 25 amino acids, about 30 amino acids, about 35 amino acids, about 40 amino acids, about 45 amino acids, about 50 amino acids, about 55 amino acids, about 60 amino acids, about 65 amino acids, about 70 amino acids, about 75 amino acids, about 80 amino acids, about 85 amino acids, about 90 amino acids, about 95 amino acids, about 100 amino acids, about 105 amino acids, about 110 amino acids, about 115 amino acids, about 120 amino acids, about 125 amino acids, about 130 amino acids, about 135 amino acids, about 140 amino acids, about 145 amino acids, about 150 amino acids, about 155 amino acids, about 160 amino acids, about 165 amino acids, about 170 amino acids, about 175 amino acids, about 180 amino acids, about 185 amino acids, about 190 amino acids, about 195 amino acids, or about 200 amino acids.

### DETAILED DESCRIPTION

Viral transduction of cells is a method of transferring a gene carried by a virus into a cell. Viral transduction is used in many cell engineering contexts, such gene editing and generation of therapeutic cells such as chimeric antigen receptor (CAR) cells. A major limitation to increasing prevalence of cell therapies has been the lack of high-purity viruses. Presented herein are fusion protein reagents including an affinity domain and a polypeptide with phase behavior. These fusion proteins can be used to purify viruses, such as lentivirus and adeno-associated virus. Advantageously, the fusion proteins presented herein are amenable to bench-scale and large-scale manufacturing protocols.

A method of purifying a virus may include contacting a virus with a fusion protein, such as mixing the fusion protein with the virus. When mixed with the fusion proteins presented herein, a virus may exhibit reduced transduction efficiency, or reduction in the number of cells contacted with the virus that are successfully infected. In some instances, this reduced transduction efficiency may be advantageous. For example, reduced transduction efficiency may improve user control of the number or type of transduced cells. However, in some instances, it may be desirable to increase transduction efficiency of viruses mixed with the fusion proteins described herein. Some methods of generically increasing transduction efficiency are known. However, it is not known whether generically increasing transduction efficiency would increase transduction efficiency of viruses mixed with fusion proteins specifically. Thus, there is a need to improve transduction efficiency for viruses mixed with the fusion proteins.

Small molecule transduction enhancers, sometimes referred to as adjuvants, are known to the art. For example, LENTIBOOST®, described, for example, in U.S. Pat. No. 10,815,498 (Anastasov et al.), is a small molecule cocktail including a poloxamer and polybrene. The amount of adjuvant used is often tightly regulated, as higher concentrations of adjuvant have been reported to decrease cell viability.

As is described herein, in the context of the fusion proteins described herein, high concentrations of a synthetic charged polymer, such as polybrene, advantageously improve transduction efficiency without decreasing cell viability. Further, the addition of other adjuvants such as poloxamers did not significantly improve transduction efficiency. Thus, the interaction between the fusion proteins, viruses, and a synthetic charged polymer (e.g., polybrene) may be mechanistically different than the interaction between virus alone with polybrene or other adjuvants.

### Fusion Proteins for Viral Purification

The fusion proteins described herein typically include an affinity domain and a polypeptide with phase behavior. Typically, the affinity domain of the fusion protein defines its application. For example, a fusion protein including an affinity domain that binds a lentivirus is typically used to bind lentivirus.

While described herein in the context of a lentiviral particle, the methods and compositions of the present disclosure are compatible with many different viral particles. For example, discussions of methods and compositions for increasing lentiviral transduction herein may apply equally to numerous other types of viral transduction. In some embodiments, a viral particle includes a retrovirus. In some embodiments, a viral particle includes a lentivirus. In some embodiments, a viral particle includes an adenovirus. In some embodiments, a viral particle includes an adeno-associated virus. In some embodiments, a viral particle includes an influenza virus.

In some embodiments, a fusion protein includes an affinity domain that binds a lentiviral particle. In some embodiments, a fusion protein includes the CR3 domain of low density lipoprotein (LDLR) and a polypeptide with phase behavior. One example of such fusion proteins is an ISOTAG® reagent, such as ISOTAG® LV (available from Donaldson Company, Inc. in Minneapolis, MN). Provided herein is a fusion protein including the CR3 domain of the low density lipoprotein (LDLR) that is at least 80% identical to the polypeptide of SEQ ID NO: 14 and a polypeptide with phase behavior that is at least 80% identical to a polypeptide of any one of SEQ ID NOS: 1, 2, or 11-13. In some embodiments, the CR3 domain of the LDLR includes a polypeptide that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of SEQ ID NO: 14. In some embodiments, the CR3 domain of the LDLR includes the polypeptide sequence of SEQ ID NO: 14. In some embodiments, the polypeptide with phase behavior includes a polypeptide that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a polypeptide of any one of SEQ ID NOS: 1, 2, or 11-13. In some embodiments, the polypeptide with phase behavior includes the polypeptide sequence of SEQ ID NO: 1. In some embodiments, the polypeptide with phase behavior includes the polypeptide sequence of SEQ ID NO: 2. In some embodiments, the polypeptide with phase behavior includes the polypeptide sequence of SEQ ID NO: 11. In some embodiments, the polypeptide with phase behavior is N-terminal to the CR3 domain of the LDLR. In some embodiments, the polypeptide with phase behavior is C-terminal to the CR3 domain of the LDLR. In some embodiments, the fusion protein includes a linker between the polypeptide with phase behavior and the CR3 domain of the LDLR. In some embodiments, the linker has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a polypeptide of any one of SEQ ID NOS: 21-38. In some embodiments, the linker includes a sequence of repeated glycine (G) residues. The linker may include at least one glycine residue and at most 30 glycine residues, such as 5 to 25 glycine residues, 10 to 20 glycine residues, or 12 to 18 glycine residues.

According to an embodiment, the composition includes one or more fusion proteins including the CR3 domain of LDLR and a polypeptide with phase behavior. In some embodiments, the CR3 domain of LDLR is a polypeptide with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 14. In some embodiments, the CR3 domain of LDLR contains from about 20 to about 39, from about 21 to about 39, from about 22 to about 39, from about 23 to about 39, from about 24 to about 39, from about 25 to about 39, from about 26 to about 39, from about 27 to about 39, from about 28 to about 39, from about 29 to about 39, from about 30 to about 39, from about 31 to about 39, from about 32 to about 39, from about 33 to about 39, from about 34 to about 39, from about 35 to about 39, from about 36 to about 39, from about 37 to about 39, or from about 38 to about 39 contiguous amino acids of SEQ ID NO: 14. In some embodiments, the CR3 domain of LDLR contains about 20, about 21, about 22, about 23, about 24, about 25, about 26, about 27, about 28, about 29, about 30, about 31, about 32, about 33, about 34, about 35, about 36, about 37, about 38, or about 39 contiguous amino acids of SEQ ID NO: 14. In some embodiments, the CR3 domain of LDLR includes from 1 to 5 modifications, from 1 to 4, from 1 to 3, of from 1 to 2 modifications compared to a polypeptide of SEQ ID NO: 14. In some embodiments, the CR3 domain of LDLR includes 1, 2, 3, 4, or 5 modifications compared to a polypeptide of SEQ ID NO: 14.

In some embodiments, the polypeptide with phase behavior has at least at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any one of SEQ ID NOS: 1, 2, 11-13, 39, and 41-42. In some embodiments, the polypeptide with phase behavior has 1, 2, 3, 4, 5, 6, 7, 8, 9, or 10 modifications compared to a polypeptide of any one of SEQ ID NOS: 1, 2, 11-13, 39, and 41-42. In some embodiments, the polypeptide with phase behavior includes from about 1 to about 5, from about 1 to about 6, from about 1 to about 7, from about 1 to about 8, from 1 to about 9, or from 1 to about 10 modifications compared to a polypeptide of any one of SEQ ID NOS: 1, 2, 11-13, 39, and 41-42.

In some embodiments, the polypeptide with phase behavior includes the amino acid sequence of SEQ ID NO: 1. In some embodiments, the polypeptide with phase behavior includes the amino acid sequence of SEQ ID NO: 2. In some embodiments, the polypeptide with phase behavior includes the amino acid sequence of SEQ ID NO: 11. In some embodiments, the polypeptide with phase behavior includes the amino acid sequence of SEQ ID NO: 12. In some embodiments, the polypeptide with phase behavior includes the amino acid sequence of SEQ ID NO: 13. In some embodiments, the polypeptide with phase behavior includes the amino acid sequence of SEQ ID NO: 39. In some embodiments, the polypeptide with phase behavior includes the amino acid sequence of SEQ ID NO: 41. In some embodiments, the polypeptide with phase behavior includes the amino acid sequence of SEQ ID NO: 42.

In some embodiments, the fusion protein is a polypeptide with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any one of SEQ ID NOS: 3-5 and 15-20. In some embodiments, the fusion protein is a polypeptide with 1, 2, 3, 4, 5, 6, 7, 8, 9, or 10 modifications compared to a polypeptide of any one of SEQ ID NOS: 3-5 and 15-20. In some embodiments, the fusion protein includes from about 1 to about 5, from about 1 to about 6, from about 1 to about 7, from about 1 to about 8, from 1 to about 9, or from 1 to about 10 modifications compared to a polypeptide of any one of SEQ ID NOS: 3-5 and 15-20.

In some embodiments, the fusion protein includes the amino acid sequence of SEQ ID NO: 3. In some embodiments, the fusion protein includes the amino acid sequence of SEQ ID NO: 5.

In some embodiments, the fusion protein includes a polypeptide with phase behavior of SEQ ID NO: 1 and the CR3 domain of LDLR (SEQ ID NO: 14). In some embodiments, the fusion protein includes a polypeptide with phase behavior of SEQ ID NO: 2 and the CR3 domain of LDLR (SEQ ID NO: 14). In some embodiments, the fusion protein includes a polypeptide with phase behavior of SEQ ID NO: 11 and the CR3 domain of LDLR (SEQ ID NO: 14). In some embodiments, the fusion protein includes a polypeptide with phase behavior of SEQ ID NO: 12 and the CR3 domain of LDLR (SEQ ID NO: 14). In some embodiments, the fusion protein includes a polypeptide with phase behavior of SEQ ID NO: 13 and the CR3 domain of LDLR (SEQ ID NO: 14). In some embodiments, the fusion protein includes a polypeptide with phase behavior of SEQ ID NO: 42 and the CR3 domain of LDLR (SEQ ID NO: 14).

In some embodiments, provided herein are nucleic acids encoding the fusion proteins described herein. In some embodiments, provided herein are nucleic acids encoding the polypeptides with phase behavior described herein. In some embodiments, the polypeptide with phase behavior is encoded by a nucleic acid of any one of SEQ ID NOS: 6-8, or a nucleic acid with at least 90%, at least 91%, at least 92%, at least 93%, at least 94%, at least 95%, at least 96%, at least 97%, at least 98%, or at least 99% identity to any one of SEQ ID NOS: 6-8. In some embodiments, the fusion protein is encoded by a nucleic acid of any one of SEQ ID NOS: 9-10, or a nucleic acid with at least 90%, at least 91%, at least 92%, at least 93%, at least 94%, at least 95%, at least 96%, at least 97%, at least 98%, or at least 99% identity to any one of SEQ ID NOS: 9-10.

In some embodiments, the fusion protein includes an N-terminal methionine. In embodiments, the fusion protein lacks an N-terminal methionine.

In some embodiments, the fusion protein includes a linker between the polypeptide with phase behavior and the CR3 domain of LDLR. In some embodiments, the linker does not interfere with the function of a fusion protein described herein. In some embodiments, the linker may adopt various secondary structures, including but not limited to α-helices, β-strands, and random coils. In some embodiments, the linker adopts an α-helix and includes an amino acid repeat of (EAAAK)ₙ (SEQ ID NO: 21) where n is an integer from 1 to 20.

In some embodiments, the linker is included of (G₄S)ₙ (SEQ ID NO: 22) where n can be an integer from 1 to 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30). In some embodiments, the polypeptide linker has a repeat of (SGGG)n (SEQ ID NO: 23), wherein n is an integer from 1 to 50 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50). In some embodiments, the polypeptide linker has a repeat of (GGGS)ₙ (SEQ ID NO: 24), wherein n is an integer from 1 to 20 (e.g., 1, 2, 3, 4, 5, 6, 7, 8, 9, 10, 11, 12, 13, 14, 15, 16, 17, 18, 19, or 20).

In some embodiments, the linker has an amino acid sequence of KESGSVSSEQLAQFRSLD (SEQ ID NO: 25). In some embodiments, the linker has an amino acid sequence of EGKSSGSGSESKST (SEQ ID NO: 26). In some embodiments, the linker only contains glycine.

In some embodiments, the peptide linker includes a protease cleavage site. In some embodiments, the protease cleavage site is a furin cleavage site.

In some embodiments, the polypeptide linker is a poly-(Gly), linker, wherein n is an integer from 1 to 30 (e.g., 1, 2, 3, 4, 5, 6, 7, 8, 9, 10, 15, 16, 17, 18, 19, 20, 21, 22, 23, 24, 25, 26, 27, 28, 29, or 30. In other embodiments, the linker is selected from the group consisting of: dipeptides, tripeptides, and quadripeptides. In some embodiments, the linker is a dipeptide selected from the group consisting of alanine-serine (AS), leucine-glutamic acid (LE), and serine-arginine (SR).

In some embodiments, the linker is selected from GKSSGSGSESKS (SEQ ID NO: 28), GSTSGSGKSSEGKG (SEQ ID NO: 29), GSTSGSGKSSEGSGSTKG (SEQ ID NO: 30), GSTSGSGKPGSGEGSTKG (SEQ ID NO: 31), EGKSSGSGSESKEF (SEQ ID NO: 32), SRSSG (SEQ ID NO: 33), and SGSSC (SEQ ID NO: 34).

In some embodiments, the linker is a self-cleaving peptide. In some embodiments, the self-cleaving peptide is a 2A peptide. 2A peptides are a class of 18-22 amino acid long peptides that induce ribosomal skipping during translation of a protein in a cell. In some embodiments, the 2A peptide is a T2A peptide having an amino acid sequence of EGRGSLLTCGDVEENPGP (SEQ ID NO: 35), a P2A peptide having an amino acid sequence of ATNFSLLKQAGDVEENPGP (SEQ ID NO: 36), an E2A peptide having an amino acid sequence of QCTNYALLKLAGDVESNPGP (SEQ ID NO: 37), or an F2A peptide having an amino acid sequence of VKQTLNFDLLKLAGDVESNPGP (SEQ ID NO: 38). In some embodiments, the 2A peptide has at least 80%, at least 85%, at least 90%, at least 95%, or at least 98% identity to any one of SEQ ID NOs. 35-38.

In some embodiments, the polypeptide with phase behavior is located N-terminal to the CR3 domain of LDLR3. In embodiments, the polypeptide with phase behavior is located C-terminal to the CR3 domain of LDLR3.

### Adjuvants and Compositions

According to an embodiment, the compositions of the present disclosure include a fusion protein reagent, one or more viral particles, and a charged polymer, where the fusion protein reagent is as described herein.

The one or more viral particles may include any virus described herein. In some embodiments, a viral particle includes a retrovirus. In some embodiments, a viral particle includes a lentivirus. In some embodiments, a viral particle includes an adenovirus. In some embodiments, a viral particle includes an adeno-associated virus. In some embodiments, a viral particle includes an influenza virus.

The compositions of the present disclosure include a polymer. As it is used herein, a polymer is a single molecule including at least two monomers covalently bound to each other. While a polymer includes a minimum of two monomers, there is no upper limit on the number of monomers in a polymer. In some embodiments, the polymer is comprised of at least two different monomer units (e.g., monomer A and monomer B). In some embodiments, the polymer has a block style architecture (e.g., AB, ABA, BAB, or the like). Typically, the compositions of the present disclosure include a charged polymer. In some embodiments, the polymer has a net charge, meaning that the summed charges of the monomers are either greater than or less than zero. The charge of the polymer typically results from the charge of individual monomers. A polymer may include monomers all having the same or a similar charge, or the polymer may include monomers having different charges. In some embodiments, the polymer has a net positive charge. In some embodiments, the polymer has a net negative charge. In some embodiments, the polymer is zwitterionic, meaning that it includes an equal number of positive and negative charges and thus has a net negative charge. A zwitterionic polymer may maintain local regions of charge. Typically, the charged polymer is a synthetic polymer. As it is used herein, a synthetic polymer is not found in nature and is chemically synthesized. However, the use of naturally occurring charged polymers is also contemplated.

In some embodiments, the synthetic polymer includes a poloxamer, such as PLURONIC®, KOLLIPHOR®, or SYNPERONIC. Poloxamers are nonionic triblock copolymers including polypropylene flanked by polyoxyethylene. In some embodiments, a poloxamer includes P181, P188, P123, P407, P124, P338, or a combination thereof.

In some embodiments, the compositions of the present disclosure include a synthetic charged polymer. In some embodiments, the synthetic charged polymer is hexadimethrine bromide, referred to herein as polybrene. Polybrene is a polymer of variable molecular weight having the chemical structure (C₁₃H₃₀Br₂N₂) n and the IUPAC name 1,5-Dimethyl-1,5-diazaundecamethylene polymethobromide. Polybrene is a cationic polymer with many uses.

In general, the amount of synthetic charged polymer may be such that the synthetic charged polymer prevents 10% or more of the blocking of cell transduction by the fusion protein reagent that would occur without the presence of the synthetic charged polymer. The synthetic charged polymer may be present in the composition in an amount that prevents 20% or more, 30% or more, 40% or more, 50% or more, 60% or more, 70% or more, or 80% or more of blocking of cell culture transduction by the fusion protein reagent. In some embodiments, the synthetic charged polymer is polybrene. The polybrene may be present in the composition in an amount that prevents 20% or more, 30% or more, 40% or more, 50% or more, 60% or more, 70% or more, or 80% or more of blocking of cell culture transduction by the fusion protein reagent.

The synthetic charged polymer (e.g., polybrene) may be present in the composition at a concentration of 10 μg/mL or more, 12 μg/mL or more, 15 μg/mL or more, 20 μg/mL or more, 25 μg/mL or more, 30 μg/mL or more, 40 μg/mL or more, or 50 μg/mL or more. The synthetic charged polymer (e.g., polybrene) may be present in the composition at a concentration of 200 μg/mL or less, 150 μg/mL or less, 100 μg/mL or less, 80 μg/mL or less, 70 μg/mL or less, 60 μg/mL or less, or 50 μg/mL or less. The synthetic charged polymer (e.g., polybrene) may be present in the composition at a concentration of 10 μg/mL to 200 μg/mL, 10 μg/mL to 100 μg/mL, 10 μg/mL to 50 μg/mL, or 12 μg/mL to 50 μg/mL. In some embodiments, the synthetic charged polymer (e.g., polybrene) is present in the composition at a concentration of about 15 μg/mL.

The synthetic charged polymer (e.g., polybrene) may be present in the composition at a molar ratio of the synthetic charged polymer to the fusion protein reagent of 4:1 or greater, 6:1 or greater, 8:1 or greater, 10:1 or greater, 15:1 or greater, 20:1 or greater, 30:1 or greater, or 50:1 or greater. The synthetic charged polymer (e.g., polybrene) may be present in the composition at a molar ratio of the synthetic charged polymer to the fusion protein reagent of 400:1 or less, 300:1 or less, 250:1 or less, 200:1 or less, 150:1 or less, 100:1 or less, 80:1 or less, 60:1 or less, or 50:1 or less. The synthetic charged polymer (e.g., polybrene) may be present in the composition at a molar ratio of the synthetic charged polymer to the fusion protein reagent of 4:1 to 400:1, 4:1 to 300:1, 6:1 to 250:1, or 10:1 to 100:1.

In some embodiments, the fusion protein reagent is ISOTAG®. In such embodiments, the synthetic charged polymer (e.g., polybrene) may be present in the composition at a molar ratio of synthetic charged polymer to the ISOTAG® of 4:1 or greater, 6:1 or greater, 8:1 or greater, 10:1 or greater, 15:1 or greater, 20:1 or greater, 30:1 or greater, or 50:1 or greater. The synthetic charged polymer (e.g., polybrene) may be present in the composition at a molar ratio of synthetic charged polymer to ISOTAG® of 400:1 or less, 300:1 or less, 250:1 or less, 200:1 or less, 150:1 or less, 100:1 or less, 80:1 or less, 60:1 or less, or 50:1 or less. The synthetic charged polymer (e.g., polybrene) may be present in the composition at a molar ratio of synthetic charged polymer to ISOTAG® of 4:1 to 400:1, 4:1 to 300:1, 6:1 to 250:1, or 10:1 to 100:1.

The synthetic charged polymer (e.g., polybrene) may be present in the composition at a weight ratio of the synthetic charged polymer to the fusion protein reagent of 1:25 or greater, 1:20 or greater, 1:15 or greater, 1:10 or greater, 1:5 or greater, or 1:1 or greater. The synthetic charged polymer (e.g., polybrene) may be present in the composition at a weight ratio of the synthetic charged polymer to the fusion protein reagent of 25:1 or less, 20:1 or less, 15:1 or less, 10:1 or less, 5:1 or less, or 1:1 or less. The synthetic charged polymer (e.g., polybrene) may be present in the composition at a weight ratio of the synthetic charged polymer to the fusion protein reagent of 25:1 to 1:25, 20:1 to 1:20, 15:1 to 1:15, or 10:1 to 1:10.

In some embodiments, the fusion protein reagent is ISOTAG®. In such embodiments, the synthetic charged polymer (e.g., polybrene) may be present in the composition at a weight ratio of synthetic charged polymer to the ISOTAG® of 1:25 or greater, 1:20 or greater, 1:15 or greater, 1:10 or greater, 1:5 or greater, or 1:1 or greater. The synthetic charged polymer (e.g., polybrene) may be present in the composition at a weight ratio of synthetic charged polymer to ISOTAG® of 25:1 or less, 20:1 or less, 15:1 or less, 10:1 or less, 5:1 or less, or 1:1 or less. The synthetic charged polymer (e.g., polybrene) may be present in the composition at a weight ratio of synthetic charged polymer to ISOTAG® of 25:1 to 1:25, 20:1 to 1:20, 15:1 to 1:15, or 10:1 to 1:10.

The composition may further include one or more cells. The one or more cells may be transduced by the one or more viral particles. The transduction efficiency of the one or more cells may be less than 90% blocked, less than 80% blocked, less than 70% blocked, less than 60% blocked, less than 50% blocked, less than 40% blocked, less than 30% blocked, or less than 20% blocked by the fusion protein reagent (e.g., ISOTAG®). The transduction efficiency of the one or more cells may be up to 90% blocked, up to 80% blocked, up to 70% blocked, up to 60% blocked, up to 50% blocked, up to 40% blocked, up to 30% blocked, up to 20% blocked, or up to 10% blocked by the fusion protein reagent (e.g., ISOTAG®). The transduction efficiency of the one or more cells may be substantially unblocked by the fusion protein reagent (e.g., ISOTAG®).

The compositions of the present disclosure may further include one or more buffers or buffering agents. Any suitable buffers typically used with cell cultures, cell purification, and/or cell transfection, and in measurement methods relating to the same, may be used. The buffers may include a binding buffer, a wash buffer, an elution buffer, a formulation buffer, or the like. In particular, the synthetic charged polymer (e.g., polybrene) may be included in an elution buffer or a formulation buffer. Examples of typical buffering agents include amino acids, such as arginine and glutamic acid, Tris, and salts, such as sodium chloride. In some embodiments, an elution buffer includes 50 mM to 200 mM arginine, 20 mM to 100 mM glutamic acid, and 20 mM to 50 mM Tris. In one or more of these embodiments, the elution buffer has a pH of 6.5. In one or more of these embodiments, the elution buffer has a conductivity of 30 milliSiemens (mS) per centimeter (cm). In some embodiments, a formulation buffer includes 20 mM to 100 mM Tris and 20 mM to 100 mM NaCl. In one or more of these embodiments, the formulation buffer has a pH of 6.8 to 7.5.

The compositions of the present disclosure may include more than one adjuvant. For example, the composition may include synthetic charged polymer and a poloxamer. The composition may include polybrene and a poloxamer.

In some embodiments, the composition is free or substantially free of poloxamer. In some embodiments, the composition is free of poloxamer.

### Methods of Use

### Methods of Transducing Cells

Provided herein is a method of transducing a cell culture including contacting the cell culture with a composition including a fusion protein reagent, one or more viral particles, and a synthetic charged polymer. The synthetic charged polymer (e.g., polybrene) to the fusion protein reagent may be present in the composition at a molar ratio of 400:1 to 4:1, as discussed above. The synthetic charged polymer (e.g., polybrene) may be included in the composition in an amount that prevents 10% or more of blocking of the cell transduction by the fusion protein reagent. The method may include contacting one or more cells with the composition. A cell culture typically includes a plurality of cells. Thus, the method may include contacting a plurality of cells with the composition.

The method may include contacting the cell with the composition for at least 12 hours, at least 24 hours, at least 36 hours, at least 48 hours, at least 72 hours, at least 96 hours, or at least 120 hours. The composition may be added to cell culture media to facilitate contact with the cell. The composition may be removed from the cell by changing the cell culture medium.

The method may include contacting the cell with the composition in a cell culture container. The cell culture container may be a bioreactor, a stir tank, a structured fixed bed, a fixed bed, a cell stack, a shake flask, a culture plate, a three-dimensional culture, or a culture plate.

Typically, the cell is an immortalized cell line, such as a cell line intended for recombinant virus production. The cell may be a HEK cell, such as a HEK293T or HEK293F cell. The cell may be a vero cell, an MDCK cell, or a HeLa cell. Cells suitable for recombinant virus production are known to the art, and any cell may be used for the methods of the present disclosure. Alternatively, the cell may be derived from a patient. For example, the cell may be derived from a patient biopsy, such as a liquid biopsy, such as a blood sample.

The synthetic charged polymer (e.g., polybrene) may be present in the composition at a concentration of 10 μg/mL or more, 12 μg/mL or more, 15 μg/mL or more, 20 μg/mL or more, 25 μg/mL or more, 30 μg/mL or more, 40 μg/mL or more, or 50 μg/mL or more. The synthetic charged polymer (e.g., polybrene) may be present in the composition at a concentration of 200 μg/mL or less, 150 μg/mL or less, 100 μg/mL or less, 80 μg/mL or less, 70 μg/mL or less, 60 μg/mL or less, or 50 μg/mL or less. The synthetic charged polymer (e.g., polybrene) may be present in the composition at a concentration of 10 μg/mL to 200 μg/mL, 10 μg/mL to 100 μg/mL, 10 μg/mL to 50 μg/mL, or 12 μg/mL to 50 μg/mL. In some embodiments, the synthetic charged polymer (e.g., polybrene) is present in the composition at a concentration of about 15 μg/mL.

The synthetic charged polymer (e.g., polybrene) may be present in the composition at a molar ratio of the synthetic charged polymer to the fusion protein reagent of 4:1 or greater, 6:1 or greater, 8:1 or greater, 10:1 or greater, 15:1 or greater, 20:1 or greater, 30:1 or greater, or 50:1 or greater. The synthetic charged polymer (e.g., polybrene) may be present in the composition at a molar ratio of the synthetic charged polymer to the fusion protein reagent of 400:1 or less, 300:1 or less, 250:1 or less, 200:1 or less, 150:1 or less, 100:1 or less, 80:1 or less, 60:1 or less, or 50:1 or less. The synthetic charged polymer (e.g., polybrene) may be present in the composition at a molar ratio of the synthetic charged polymer to the fusion protein reagent of 4:1 to 400:1, 4:1 to 300:1, 6:1 to 250:1, or 10:1 to 100:1.

The synthetic charged polymer (e.g., polybrene) may be present in the composition at a weight ratio of the synthetic charged polymer to the fusion protein reagent of 1:25 or greater, 1:20 or greater, 1:15 or greater, 1:10 or greater, 1:5 or greater, or 1:1 or greater. The synthetic charged polymer (e.g., polybrene) may be present in the composition at a weight ratio of the synthetic charged polymer to the fusion protein reagent of 25:1 or less, 20:1 or less, 15:1 or less, 10:1 or less, 5:1 or less, or 1:1 or less. The synthetic charged polymer (e.g., polybrene) may be present in the composition at a weight ratio of the synthetic charged polymer to the fusion protein reagent of 25:1 to 1:25, 20:1 to 1:20, 15:1 to 1:15, or 10:1 to 1:10.

The synthetic charged polymer (e.g., polybrene) may be present in the composition in an amount that prevents 20% or more, 30% or more, 40% or more, 50% or more, 60% or more, 70% or more, or 80% or more of blocking of the cell culture transduction by the fusion protein reagent.

In some embodiments, a method of transducing a cell culture is a method of increasing transduction efficiency of the cell culture. In particular, a method may increase transduction efficiency relative to a comparable method where the synthetic charged polymer (e.g., polybrene) is absent. In some embodiments, a method of the present disclosure increases transduction efficiency by 20% or more, 30% or more, 40% or more, 50% or more, 60% or more, 70% or more, or 80% or more relative to a comparable method where synthetic charged polymer (e.g., polybrene) is absent. In some embodiments, the method of transducing a cell culture is a method of matching transduction efficiency to a comparable method not including a fusion protein. As is described herein, addition of a fusion protein of the present disclosure may decrease transduction efficiency of a viral preparation. Thus, the adjuvants described herein, specifically synthetic charged polymer (e.g., polybrene), may advantageously restore transduction efficiency of a viral preparation to the level achieved without addition of a fusion protein.

In some embodiments, the method of transducing a cell culture is a method of improving lentiviral transduction efficiency. The method of improving lentiviral transduction efficiency includes contacting a fusion protein described with a cell. The method of improving lentiviral transduction efficiency may include adding a fusion protein to a cell culture. The fusion protein may be administered simultaneously with (e.g., within 15 minutes of administering) a lentivirus. In some embodiments, the fusion protein is administered within 15 minutes, within 30 minutes, within 45 minutes, within an hour, within 2 hours, within 3 hours, within 4 hours, within 5 hours, within 6 hours, within 7 hours, within 8 hours, within 9 hours, within 10 hours, within 11 hours, within 12 hours, within 13 hours, within 14 hours, within 15 hours, within 16 hours, within 17 hours, within 18 hours, within 19 hours, within 20 hours, within 21 hours, within 22 hours, within 23 hours, or within 24 hours of administering the lentivirus. In some embodiments, the fusion protein is administered to the cell before lentivirus is administered to the cell. In some embodiments, the fusion protein is administered to the cell after lentivirus is administered to the cell. In some embodiments, the fusion protein has a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOS: 3-5 or 15-20. In some embodiments, the fusion protein is an ISOTAG® LV (available from Donaldson Company, Inc. in Minneapolis, MN).

In some embodiments, the method of improving lentiviral transduction efficiency includes administering a polypeptide with phase behavior to a cell. In some embodiments, the polypeptide with phase behavior is administered simultaneously with (e.g., within 15 minutes of administering) a lentivirus. In some embodiments, the polypeptide with phase behavior is administered within 15 minutes, within 30 minutes, within 45 minutes, within an hour, within 2 hours, within 3 hours, within 4 hours, within 5 hours, within 6 hours, within 7 hours, within 8 hours, within 9 hours, within 10 hours, within 11 hours, within 12 hours, within 13 hours, within 14 hours, within 15 hours, within 16 hours, within 17 hours, within 18 hours, within 19 hours, within 20 hours, within 21 hours, within 22 hours, within 23 hours, or within 24 hours of administering lentivirus. In some embodiments, the polypeptide with phase behavior is administered to the cell before lentivirus is administered to the cell. In some embodiments, the polypeptide with phase behavior is administered to the cell after lentivirus is administered to the cell. In some embodiments, the polypeptide with phase behavior has a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOS: 1, 2, or 11-13.

In some embodiments, lentiviral transduction efficiency (or the percentage of cells that are infected with lentivirus) is evaluated by flow cytometry. For example, in some embodiments, a lentivirus encodes a fluorescent protein, such as green fluorescent protein (GFP). The lentiviral transduction efficiency is the percentage of cells that express the fluorescent protein. As another example, in some embodiments, a lentivirus encodes a protein that can be detected on the cell surface, such as CD19. Cells transduced with lentivirus encoding the cell surface protein may be incubated with a fluorescent antibody that binds to the cell surface protein. The lentiviral transduction efficiency is the percentage of cells with bound fluorescent antibody.

In some embodiments, lentiviral transduction efficiency (or the percentage of cells that are infected with lentivirus) is evaluated by cell imaging or using a plaque assay.

In some embodiments, the methods described herein provide improved lentiviral transduction efficiency compared to an alternative method. In embodiments, the alternative method includes a lentiviral transduction method which does not administer the fusion protein or the polypeptide exhibiting phase behavior with synthetic charged polymer (e.g., polybrene). In some embodiments, the alternative method is a conventional method for improving lentiviral transduction.

In some embodiments, a method of improving viral transduction includes temporal control of viral transduction. In some embodiments, it may be desirable to titrate the infectivity (e.g., transduction efficiency) of a set amount of viral material. As is described herein, the fusion protein reagents of the present disclosure may reduce transduction efficiency of a virus. Further, the synthetic polymers described herein may counteract this reduction in transduction efficiency, ultimately increasing transduction efficiency in the context of a virus mixed with a fusion protein. Thus, in combination, the fusion protein of the present disclosure may be used to spatiotemporally control the effective titer the infectivity of virus material.

In some embodiments, a method includes contacting a virus-producing cell with a fusion protein. During recombinant production of some viruses, viral particles are released into the culture media, which viral particles may subsequently infect the virus-producing cells. In some embodiments, infection of virus-producing cells ultimately decreases total virus production in the batch. Thus, it may be beneficial to prevent infection of virus-producing cells by viruses within the culture media.

In some embodiments, the method for improving lentiviral transduction efficiency includes administering a polypeptide with phase behavior with an amino acid sequence that is at least 90%, at least 91%, at least 92%, at least 93%, at least 94%, at least 95%, at least 96%, at least 97%, at least 98%, at least 99%, or 100% identical to SEQ ID NO: 4. In embodiments, administering a polypeptide with phase behavior with an amino acid sequence that is at least 90%, at least 91%, at least 92%, at least 93%, at least 94%, at least 95%, at least 96%, at least 97%, at least 98%, at least 99%, or 100% identical to SEQ ID NO: 4 improves lentiviral transduction efficiency by at least 90% compared to an alternative method.

### Methods of Measuring Viral Titer

According to an embodiment, a method of measuring a concentration of transducing units in a viral preparation includes contacting one or more cells with one or more viral particles and a fusion protein reagent to form an intermediate composition. The fusion protein reagent includes an affinity domain and a polypeptide with phase behavior. The intermediate composition includes a cationic polymer such as polybrene at a concentration of 10 μg/mL or greater, such as 11 μg/mL or greater, 12 μg/mL or greater, 13 μg/mL or greater, 14 μg/mL or greater, 15 μg/mL or greater, 16 μg/mL or greater, or 20 μg/mL or greater. The one or more viral particles include a genetic payload, The method further includes incubating the cell for an incubation period of 12 hours or longer, such as 24 hours or longer, 36 hours or longer, 42 hours or longer, 48 hours or longer, 60 hours or longer, 72 hours or longer, or 84 hours or longer. The method further includes detecting infection in the one or more cells and calculating the concentration of transducing units based on the number of infected cells. The fusion protein reagent is discussed above. The fusion protein reagent may be an ISOTAG® reagent.

The genetic payload of the viral particles may include a detectable marker. The detectable marker may include a fluorescent protein. The detectable marker may include a protein that can be detected on the surface of a cell. The detectable marker may include an affinity tag, such as a His tag, a myc tag, or a FLAG tag. Detecting infection may include flow cytometry, fluorescent microscopy, cell imaging, a plaque assay, or a combination of two or more thereof. The method may include quantifying the number of viral particles. The quantifying may include enzyme-linked immunosorbent assay (ELISA). The method may further include quantifying the number of genomes in the one or more viral particles to provide a concentration of viral genomes. The method may further include using quantitative real-time PCR (qRT-PCR).

The methods of the present disclosure may include contacting two or more cell cultures with two or more dilutions of the viral preparation. Each of the contacting of the cell cultures with the viral preparations may include contacting the cell cultures with the compositions as discussed herein, including a fusion protein agent and synthetic charged polymer (e.g., polybrene).

The method may further include comparing the concentration of viral genomes and the viral titer, where the concentration of viral genomes is within 10% of the concentration of transducing units. The concentration of viral genomes may be within 20%, within 30%, within 40%, within 50%, within 60%, within 70%, within 80%, or within 90% of the concentration of transducing units.

The concentrations and relative amounts of synthetic charged polymer (e.g., polybrene) and fusion protein reagent in the composition are as discussed above. Further, the additional components, including buffering agents and adjuvants are also as discussed above.

### Kits

The components for the compositions and methods described in the present disclosure may be provided as a kit. According to an embodiment, a kit for transducing a cell may include a fusion protein reagent having an affinity domain and a polypeptide with phase behavior; one or more viral particles; a synthetic charged polymer; and instructions for transducing the cell by contacting the cell with a composition including the fusion protein reagent, the one or more viral particles, and the synthetic charged polymer, where a molar ratio of synthetic charged polymer to the fusion protein reagent is from 400:1 to 4:1. The instructions may further include instructions to prepare the composition using the fusion protein reagent, the one or more viral particles, and the synthetic charged polymer.

According to an embodiment, a kit for transducing a cell may include a fusion protein reagent having an affinity domain and a polypeptide with phase behavior; one or more viral particles; polybrene; and instructions for transducing the cell by contacting the cell with a composition including the fusion protein reagent, the one or more viral particles, and the polybrene, where a molar ratio of polybrene to the fusion protein reagent is from 400:1 to 4:1. The instructions may further include instructions to prepare the composition using the fusion protein reagent, the one or more viral particles, and the polybrene.

In some embodiments, a kit for purifying a virus may include a fusion protein reagent having an affinity domain and a polypeptide with phase behavior; a binding buffer; a wash buffer; an elution buffer; a formulation buffer; synthetic charged polymer (e.g., polybrene); and instructions for purifying the virus by using the fusion protein reagent, the binding buffer, the wash buffer, the elution buffer, and the polybrene.

In some embodiments, a kit for purifying a virus may include an ISOTAG® reagent; a binding buffer; a wash buffer; an elution buffer; a formulation buffer; synthetic charged polymer (e.g., polybrene); and instructions for purifying the virus by using the ISOTAG® reagent, the binding buffer, the wash buffer, the elution buffer, and the synthetic charged polymer.

The components of the kits and their concentrations in the composition are described above.

### ILLUSTRATIVE EMBODIMENTS

The following are a list of illustrative embodiments according to the present disclosure.

Embodiment 1 is a composition including a fusion protein reagent, one or more viral particles, and synthetic charged polymer, wherein the fusion protein reagent includes an affinity domain and a polypeptide with phase behavior, and wherein a molar ratio of the synthetic charged polymer to the fusion protein reagent is 400:1 to 4:1.

Embodiment 2 is the composition of embodiment 1, wherein the synthetic charged polymer includes a cationic polymer.

Embodiment 3 is the composition of embodiment 1 or 2, wherein the synthetic charged polymer includes polybrene.

Embodiment 4 is the composition of any one of embodiments 1 to 3, wherein the fusion protein reagent is an ISOTAG® reagent.

Embodiment 5 is the composition of any one of embodiments 1 to 4, wherein a weight ratio of the synthetic charged polymer to the fusion protein reagent is from 25:1 to 1:25.

Embodiment 6 is the composition of any one of embodiments 1 to 6, wherein a concentration of synthetic charged polymer in the composition is 10 μg/mL or more.

Embodiment 7 is the composition of any one of embodiments 1 to 7, further including one or more cells.

Embodiment 8 is a method of transducing one or more cells including: contacting the one or more cells with a composition including a fusion protein reagent, one or more viral particles, and synthetic charged polymer, wherein the fusion protein reagent includes an affinity domain and a polypeptide with phase behavior, and wherein a molar ratio of the synthetic charged polymer to the fusion protein reagent is 400:1 to 4:1.

Embodiment 9 is the method of embodiment 8, wherein the synthetic charged polymer includes polybrene.

Embodiment 10 is the method of embodiment 8 or 9, wherein the fusion protein reagent is an ISOTAG® reagent.

Embodiment 11 is the method of any one of embodiments 8 to 10, wherein a weight ratio of the synthetic charged polymer to the fusion protein reagent is from 25:1 to 1:25.

Embodiment 12 is the method of any one of embodiments 8 to 11, further including calculating transduction efficiency, wherein the transduction efficiency is higher than one or more cells contacted with a comparable composition lacking a synthetic charged polymer.

Embodiment 13 is a method of transducing one or more cells including: contacting the one or more cells with a composition including: a fusion protein reagent including an affinity domain and a polypeptide with phase behavior; one or more viral particles; and a synthetic charged polymer, wherein the synthetic charged polymer is included in the composition in an amount that prevents 10% or more of blocking of cell transduction by the fusion protein reagent.

Embodiment 14 is the method of embodiment 13, wherein the synthetic charged polymer includes polybrene.

Embodiment 15 is the method of embodiment 13 or 14, wherein the fusion protein reagent is an ISOTAG® reagent.

Embodiment 16 is the method of any one of embodiments 13 to 15, wherein a molar ratio of the synthetic charged polymer to the fusion protein reagent is 400:1 to 4:1.

Embodiment 17 is the method of any one of embodiments 13 to 16, wherein a weight ratio of the synthetic charged polymer to the fusion protein reagent is from 25:1 to 1:25.

Embodiment 18 is the method of any one of embodiments 13 to 17, wherein the synthetic charged polymer prevents 20% or more, 30% or more, 40% or more, 50% or more, 60% or more, 70% or more, or 80% or more of blocking of cell culture transduction by the fusion protein reagent.

Embodiment 19 is the method of any one of embodiments 13 to 18, wherein the synthetic charged polymer prevents up to 90% of blocking of cell transduction by the fusion protein reagent.

Embodiment 20 is a method of measuring a concentration of transducing units in a viral preparation including: contacting one or more cells with one or more viral particles and a fusion protein reagent to form an intermediate composition, the fusion protein reagent including an affinity domain and a polypeptide with phase behavior, the intermediate composition including a synthetic charged polymer at a concentration of 10 μg/mL or greater, and the one or more viral particles including a genetic payload; incubating the cell for 12 hours or longer; detecting infection in the one or more cells; and calculating the concentration of transducing units based on the number of infected cells.

Embodiment 21 is the method of embodiment 20, wherein the synthetic charged polymer includes polybrene.

Embodiment 22 is the method of embodiment 20 or 21, wherein the fusion protein reagent is an ISOTAG® reagent.

Embodiment 23 is the method of any one of embodiments 20 to 22, wherein the genetic payload includes a detectable marker.

Embodiment 24 is the method of embodiment 23, wherein the detectable marker includes a fluorescent protein.

Embodiment 25 is the method of any one of embodiments 20 to 24, wherein detecting infection includes flow cytometry.

Embodiment 26 is the method of any one of embodiments 20 to 25, wherein detecting infection includes fluorescent microscopy, cell imaging, or a plaque assay.

Embodiment 27 is the method of any one of embodiments 20 to 26, wherein contacting a cell with one or more viral particles includes contacting two or more cell cultures with two or more dilutions of the viral preparation.

Embodiment 28 is the method of any one of embodiments 20 to 27, further including quantifying number of genomes in the one or more viral particles to provide a concentration of viral genomes.

Embodiment 29 is the method of embodiment 28, wherein the quantifying includes quantitative real-time PCR (qRT-PCR).

Embodiment 30 is the method of any one of embodiments 20 to 29, further including quantifying the number of viral particles.

Embodiment 31 is the method of embodiment 30, wherein the quantifying includes enzyme-linked immunosorbent assay (ELISA).

Embodiment 32 is the method of embodiment 30 or 31, further including comparing percent recovery of viral particles and viral titer, wherein the percent recovery of viral particles is within 10% of the transducing units.

Embodiment 33 is the method of embodiment 32, wherein the intermediate composition includes the synthetic charged polymer at a concentration of 12 μg/mL or greater, or about 15 μg/mL.

Embodiment 34 is the method of embodiment 32 or 33, wherein the intermediate composition includes the synthetic charged polymer at a concentration of 200 μg/mL or less, 150 μg/mL or less, or 100 μg/mL or less.

Embodiment 35 is the method of any one of embodiments 32 to 34, wherein a weight ratio of the synthetic charged polymer to the fusion protein reagent is from 25:1 to 1:25.

Embodiment 36 is the method of any one of embodiments 32 to 35, wherein a molar ratio of the synthetic charged polymer to the fusion protein reagent is from 400:1 to 4:1.

Embodiment 37 is a kit for transducing a cell including: a fusion protein reagent including an affinity domain and a polypeptide with phase behavior; one or more viral particles; a synthetic charged polymer; and instructions for transducing the cell by contacting the cell with a composition including the fusion protein reagent, the one or more viral particles, and the synthetic charged polymer, wherein a molar ratio of the synthetic charged polymer to the fusion protein reagent is from 400:1 to 4:1.

Embodiment 38 is the kit of embodiment 37, wherein the fusion protein reagent is an ISOTAG® reagent.

Embodiment 39 is the kit of embodiment 38, wherein the instructions further include instructions to prepare the composition using the fusion protein reagent, the one or more viral particles, and the synthetic charged polymer.

Embodiment 40 is a kit for purifying a virus including: an ISOTAG® reagent; a binding buffer; a wash buffer; an elution buffer; a formulation buffer; a synthetic charged polymer; and instructions for purifying the virus by using the ISOTAG® reagent, the binding buffer, the wash buffer, the elution buffer, and the synthetic charged polymer.

Embodiment 41 is the kit of any one of embodiments 37 to 40, wherein the synthetic charged polymer includes polybrene.

Embodiment 42 is the kit of any one of embodiments 37 to 41, wherein the formulation buffer or the elution buffer includes the synthetic charged polymer at a concentration of 10 μg/mL or more.

Embodiment 43 is the kit of any one of embodiments 37 to 42, wherein the instructions include instructions to add the synthetic charged polymer to the ISOTAG® reagent at a weight ratio of the synthetic charged polymer to viral particles of 25:1 to 1:25.

Embodiment 44 is the kit of any one of embodiments 37 to 43, wherein the instructions include instructions to add the synthetic charged polymer to the ISOTAG® reagent at a molar ratio of 400:1 to 4:1.

Embodiment 45 is a method of controlling viral transduction of a cell, the method including: contacting the cell with a first composition including one or more viral particles and a fusion protein reagent, causing down-regulation of viral transduction of the cell; and contacting the cell with a second composition including the one or more viral particles and a synthetic charged polymer, causing up-regulation of viral transduction of the cell.

Embodiment 46 is the method of embodiment 45, wherein the fusion protein reagent includes a fusion protein with an affinity domain and a polypeptide with phase behavior.

Embodiment 47 is the method of embodiment 45 or 46, wherein the cell is a virus-producing cell capable of producing the one or more viral particles.

Embodiment 48 is the method of any one of embodiments 45 to 47, wherein the method further includes causing the cells to produce the one or more viral particles prior to the contacting of the cell with the first composition.

Embodiment 49 is a method of controlling transduction of a virus-producing cell, the method including contacting the virus-producing cell with a fusion protein.

Embodiment 50 is the method of embodiment 49, further including contacting the cell with one or more reagents encoding one or more viral components.

Embodiment 51 is the method of any one of embodiments 45 to 50, wherein the fusion protein includes an ISOTAG® reagent.

Embodiment 52 is the method of any one of embodiments 45 to 51, wherein the synthetic charged polymer includes polybrene.

Embodiment 53 is the method of any one of embodiments 45 to 51, wherein a molar ratio of the synthetic charged polymer to the fusion protein reagent is 400:1 to 4:1.

Embodiment 54 is the method of any one of embodiments 45 to 53, wherein a weight ratio of the synthetic charged polymer to the fusion protein reagent is from 25:1 to 1:25.

Embodiment 55 is the method of any one of embodiments 45 to 54, wherein the synthetic charged polymer prevents 20% or more, 30% or more, 40% or more, 50% or more, 60% or more, 70% or more, or 80% or more of blocking of cell culture transduction by the fusion protein reagent.

Embodiment 56 is the method of any one of embodiments 45 to 55, wherein the synthetic charged polymer prevents up to 90% of blocking of cell transduction by the fusion protein reagent.

### EXAMPLES

The present invention is illustrated by the following examples. It is to be understood that the particular examples, materials, amounts, and procedures are to be interpreted broadly in accordance with the scope and spirit of the invention as set forth herein.

### Lentivirus Preparation

HEK293T cells were seeded 24 hours before transfection. On day 1, cells were transfected with lentiviral plasmids, CMV promoter, and the GFP reporter. Lentiviral particles were released from the cells into the supernatant and collected 24 hours to 72 hours post transfection. For adherent HEK293T cultures, supernatant was collected from the cell culture. For suspension HEK293F cultures, the entire sample was centrifuged to pellet cells, upon which the supernatant containing the lentiviral vectors was removed. Supernatant containing lentiviral vectors was either stored at −80 °C or used fresh for experiments.

All lentiviruses used in the Examples described herein encoded green fluorescent protein (GFP) under control of a strong promoter.

### Affinity Liquid Phase Separation-Centrifugation (ALPS-CF) Capture Protocol

Lentiviral vectors (LVV) described above were mixed with 0 μM to 20 μM ISOTAG® LV for 5 minutes to 60 minutes on ice to bind the ISOTAG® LV to the LVVs. NaCl was added at 0.5 M to 1.5 M to trigger ISOTAG® LV droplet formation. Sometimes the samples are heated to 37 °C for this step. The ISOTAG® LV, LVV, NaCl mixture mixed for 5 minutes to 60 minutes before being centrifuged at 500×g to 4000×g. The resulting pellet contained concentrated LVV bound to ISOTAG® LV.

### Affinity Liquid Phase Separation-Tangential Flow Filtration (ALPS-TFF) LV Purification Protocol

Lentiviral vectors described above were mixed with 0 μM to 20 μM ISOTAG® LV for 1 to 12 hours at 4 °C to bind the ISOTAG® LV to the LVVs. NaCl was added at 0.5 M to 1.5 M to trigger ISOTAG® LV droplet formation. Sometimes the samples are heated to 37 °C for this step. The ISOTAG® LV, LVV, NaCl mixture mixed for 5 minutes to 60 minutes before being concentrated and washed on TFF1, a microfiltration TFF step in permeate control mode. The concentrated ISOTAG® LV, LVV, NaCl mixture was then diluted in ice cold elution buffer and concentrated on TFF2 on ice, a microfiltration TFF step in permeate control mode. In TFF2 the ISOTAG® LV was washed through the filter into the waste. The resulting concentrated sample of purified LVV contained 0 μM to 50 μM of residual ISOTAG® LV.

### Transduction Protocol

HEK293T cells to be transduced were plated in a 96-well plate. Cell health and adherence were confirmed before starting transduction.

To begin the transduction, lentiviral material, ISOTAG® LV (available from Donaldson Company, Inc. in Minneapolis, MN) at a stock concentration of 250 μM to 300 μM, and polybrene at a stock concentration of 10 mg/mL were thawed on ice. polybrene was diluted in sterile water to a working concentration of 1,000 μg/mL. Other adjuvants including LENTIBOOST® and poloxamer 188 (P188) were thawed. The desired amount of lentiviral material was added to each well. Well volume was normalized to 106 μL using culture media. After lentiviral material had been added, ISOTAG® LV was added to wells to achieve a desired concentration. After ISOTAG® LV was added to wells, polybrene was added to wells to achieve a desired concentration. The cell culture plate was swirled gently to mix the added components.

Cells were incubated at 37 °C and 5% CO₂ for 48 hours. Following incubation, cells were analyzed, for example, using fluorescence microscopy and/or flow cytometry.

### FACS Protocol

First, transduction was confirmed by observing cells expressing GFP using fluorescence microscopy. To harvest the cells for flow cytometry, cell culture supernatant was removed from the dish with an aspirator pipette. 50 μL of trypsin was added to each well to evenly coat cells. Cells were incubated at 37 °C for five minutes. Following incubation, cells were detached by lightly tapping the bottom of the cell culture plate. 50 μL of flow buffer (4% fetal bovine serum (FBS) in 1×PBS) was added to each well to inactivate the trypsin. Cells were triturated to produce a single cell solution. Samples were transferred to a 96-well V-bottom plate and analyzed using the flow cytometer.

To analyze flow cytometry data, cells were gated using front and side scatter to isolate single cells. Non-transduced cells were analyzed to determine the level of background fluorescence. A GFP positive gate was set using the background fluorescence level. The percentage of GFP positive cells in each sample was measured.

### Example 1

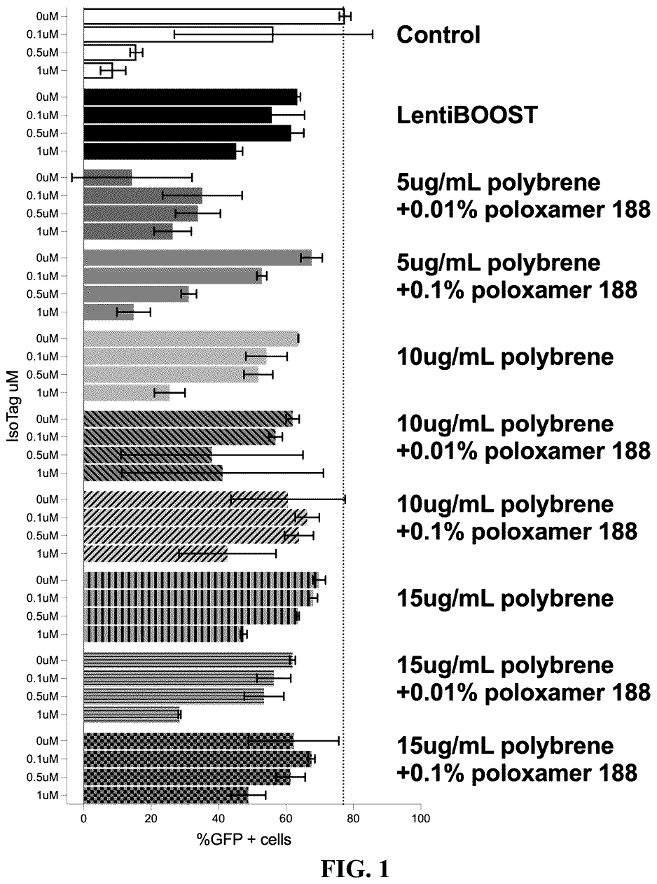

Cells were transduced with lentiviral material encoding GFP as described above. Cell transduction efficiency was measured using FACS as described above. The transduction efficiency observed in each condition, quantified as the percentage of GFP positive cells in each culture, is represented in FIG. 1.

For each sample, 6 μL of LV was added to 100 μL of cell culture. The concentration of ISOTAG®™ LV added was 0 μM-1 μM. Once the 6 μL of LV (containing ISOTAG®™ LV) was added to the cell culture media, the adjuvants described in FIG. 1 were added. The mixture incubated with the cells for 48 hours before being analyzed using FACS as described above. In samples from ALPS-CF, the expected concentration of ISOTAG®™ LV on cells when mixed with concentrated LV is 0.2-0.5 M. Without polybrene, this concentration was expected to block LV transduction.

It was observed that the ISOTAG® LV alone (control) efficiently blocked transduction at concentrations greater than 0.5 μM. The addition of LENTIBOOST® reversed at least 50% of the blocking caused by the ISOTAG® LV in all groups. It was observed that the increased concentration of polybrene alone (15 μg/mL) had almost 100% reversal of blocking in samples with 0.1 μM to 0.5 μM of ISOTAG® LV compared to the control without any polybrene present. Additionally, it was observed that 5 μg/mL polybrene was too low to significantly reverse ISOTAG® LV transduction blocking. It was observed the addition of poloxamer 188 had no significant effect on ISOTAG® LV behavior. It was observed that cells treated with 10-15 μg/mL polybrene with or without poloxamer 188 improved transduction when compared to cells without adjuvant.

### Example 2

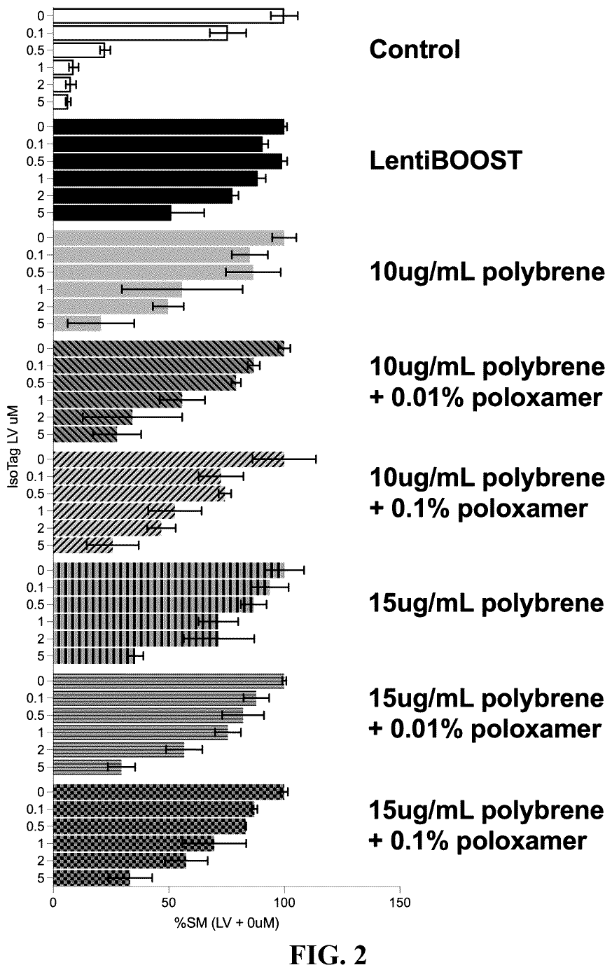

Cells were transduced with lentiviral material encoding GFP as described above. Cell transduction efficiency was measured using FACS as described above. The transduction efficiency observed in each condition, quantified as the percentage of GFP positive cells in each culture, is represented in FIG. 2.

For each sample, 6 μL of LV was added to 100 μL of cell culture. The concentration of ISOTAG® LV added was 0-5 μM. Once the 6 μL of LV (containing ISOTAG® LV) was added to the cell culture media, the adjuvants described in FIG. 2 were added. The mixture incubated with the cells for 48 hours before being analyzed using FACS as described above.

Increased transduction efficiency was observed across all samples containing ISOTAG® LV when at least 10 μg/mL of polybrene is present compared to controls. Better recovery was observed with 15 μg/mL polybrene for samples containing 0 μM to 2 μM. It was observed that the addition of poloxamer 188 to samples including 15 μg/mL of polybrene did not increase transduction efficiency. Rather, 15 μg/mL polybrene reversed blocking caused by ISOTAG® LV comparable to samples of cells treated with LENTIBOOST® and ISOTAG® LV.

### Example 3

Samples were collected from ALPS-TFF experiments as described above. Cells were transduced with lentiviral material encoding GFP as described above. Cell transduction efficiency was measured using FACS as described above. The transduction efficiency observed in each condition, quantified as the percentage of GFP positive cells in each culture, is represented in Table 1 and Table 2.

TABLE 1
Transducing units observed with and without polybrene.

| Sample | TU | TU + Polybrene | % Change |
|---|---|---|---|
| LV control | 3.21E+08 | 3.47E+08 | +8% |
| LV + ISOTAG ® | 9.53E+07 | 1.29E+08 | +35% |
| TFF1 Waste | 2.63E+07 | 2.11E+07 | −20% |
| TFF1 Concentrate | 1.92E+08 | 4.33E+08 | +125% |
| (also contains ISOTAG ®) | | | |
| TFF2 Waste | 1.12E+07 | 3.90E+07 | +248% |
| (also contains ISOTAG ®) | | | |
| TFF2 Concentrate | 3.52E+07 | 7.04E+07 | +100% |
| (also contains ISOTAG ®) | | | |

TABLE 2
Transducing units observed with and without LENTIBOOST ®.

| | | TU + | % |
|---|---|---|---|
| Sample | TU | LENTIBOOST ® | Change |
| LV control | 8.58E+08 | 2.49E+09 | +190% |
| LV + ISOTAG ® | 1.63E+09 | 1.27E+09 | −22% |
| TFF1 Waste | 1.18E+09 | 1.34E+09 | +13% |
| TFF1 Concentrate | 1.90E+09 | 1.19E+09 | −37% |
| (also contains ISOTAG ®) | | | |
| TFF2 Waste | 1.60E+07 | 1.90E+07 | +18% |
| (also contains ISOTAG ®) | | | |
| TFF2 Concentrate | 4.28E+08 | 4.88E+08 | +14% |
| (also contains ISOTAG ®) | | | |

It was observed that samples gathered from ALPS-TFF containing ISOTAG® LV (bold) experienced a significant transduction enhancement, or reversal of blocking, when 15 μg/mL of polybrene were added to the samples for transduction. This effect was not noted in samples without ISOTAG® LV when 15 μg/mL of polybrene was added. Conversely, samples from a separate ALPS-TFF experiment did not experience a significant transduction enhancement when 1:50 LENTIBOOST® was added to samples with or without ISOTAG® LV. Interestingly, the initial control LV sample did exhibit enhanced transduction when 1:50 LENTIBOOST® was added. Without wishing to be bound by theory, this suggests that the mechanism by which LENTIBOOST® improves transduction differs from the mechanism by which polybrene reverses blocking from ISOTAG® LV.

### Example 4

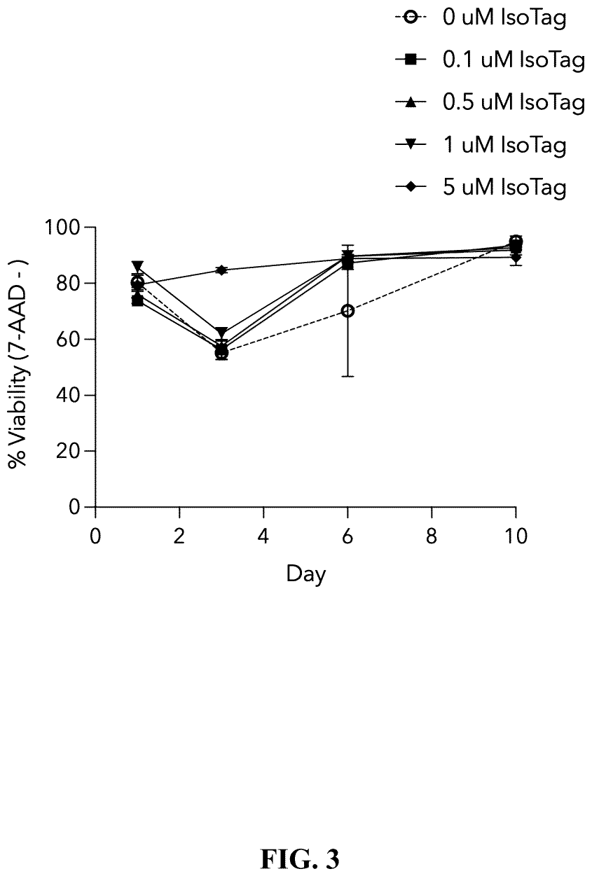

HEK293T cells were seeded 24 hours before experimentation and viability was recorded. On day 1, media was replaced with fresh media containing 0 μM to 5 μM ISOTAG® LV and samples were taken for viability testing. The cells were incubated with the ISOTAG® LV containing media for up to 10 days with samples taken throughout for viability testing. For viability testing, media was removed and the cells were washed with 1×PBS. Trypsin was used to detach the adherent cells from the plate. The cells were incubated with 7-AAD dye, which binds to DNA and can be measured using flow cytometry. Cells negative for 7-AAD dye were considered viable. Cell viability is depicted in FIG. 3. It was observed that cell viability was not significantly affected by treatment with ISOTAG® LV (FIG. 3). From this data, it was concluded any change in infectious titers was due to ISOTAG®. fusion protein, or polybrene addition, or combinations thereof, rather than changes in cell viability.

What is claimed is:

1. A composition comprising a fusion protein reagent, one or more viral particles, and synthetic charged polymer, wherein the fusion protein reagent comprises an affinity domain and a polypeptide with phase behavior, and wherein a molar ratio of the synthetic charged polymer to the fusion protein reagent is 400:1 to 4:1.

2. The composition of claim 1, wherein the synthetic charged polymer comprises a cationic polymer.

3. The composition of claim 1, wherein the synthetic charged polymer comprises polybrene.

4. The composition of claim 1, wherein the fusion protein reagent is an ISOTAG® reagent.

5. The composition of claim 1, wherein a weight ratio of the synthetic charged polymer to the fusion protein reagent is from 25:1 to 1:25.

6. The composition of claim 1, wherein a concentration of synthetic charged polymer in the composition is 10 μg/mL or more.

7. A method of transducing one or more cells comprising:
contacting the one or more cells with a composition comprising a fusion protein reagent, one or more viral particles, and synthetic charged polymer, wherein the fusion protein reagent comprises an affinity domain and a polypeptide with phase behavior, and wherein a molar ratio of the synthetic charged polymer to the fusion protein reagent is 400:1 to 4:1.

8. The method of claim 7, wherein the synthetic charged polymer comprises polybrene.

9. The method of claim 7, wherein the fusion protein reagent is an ISOTAG® reagent.

10. The method of claim 7, further comprising calculating transduction efficiency, wherein the transduction efficiency is higher than one or more cells contacted with a comparable composition lacking a synthetic charged polymer.

11. A method of transducing one or more cells comprising:
contacting the one or more cells with a composition comprising:
a fusion protein reagent comprising an affinity domain and a polypeptide with phase behavior;
one or more viral particles; and
a synthetic charged polymer, wherein the synthetic charged polymer is included in the composition in an amount that prevents 10% or more of blocking of cell transduction by the fusion protein reagent.

12. The method of claim 11, wherein the synthetic charged polymer comprises polybrene.

13. The method of claim 11, wherein the fusion protein reagent is an ISOTAG® reagent.

14. The method of claim 11, wherein the synthetic charged polymer prevents 20% or more, 30% or more, 40% or more, 50% or more, 60% or more, 70% or more, or 80% or more of blocking of cell culture transduction by the fusion protein reagent.

15. A method of measuring a concentration of transducing units in a viral preparation comprising:
contacting one or more cells with one or more viral particles and a fusion protein reagent to form an intermediate composition, the fusion protein reagent comprising an affinity domain and a polypeptide with phase behavior, the intermediate composition comprising a synthetic charged polymer at a concentration of 10 μg/mL or greater, and the one or more viral particles comprising a genetic payload;
incubating the cell for 12 hours or longer;
detecting infection in the one or more cells; and
calculating the concentration of transducing units based on the number of infected cells.

16. The method of claim 15, wherein the synthetic charged polymer comprises polybrene.

17. The method of claim 15, wherein the fusion protein reagent is an ISOTAG® reagent.

18. The method of claim 15, further comprising quantifying number of genomes in the one or more viral particles to provide a concentration of viral genomes.

19. The method of claim 15, further comprising quantifying the number of viral particles.

20. The method of claim 19, further comprising comparing percent recovery of viral particles and viral titer, wherein the percent recovery of viral particles is within 10% of the transducing units.

21. A kit for purifying a virus comprising:
an ISOTAG® reagent;
a binding buffer;
a wash buffer;
an elution buffer;
a formulation buffer;
a synthetic charged polymer; and
instructions for purifying the virus by using the ISOTAG® reagent, the binding buffer, the wash buffer, the elution buffer, and the synthetic charged polymer.

22. The kit of claim 21, wherein the synthetic charged polymer comprises polybrene.

23. The kit of claim 21, wherein the formulation buffer or the elution buffer comprises the synthetic charged polymer at a concentration of 10 μg/mL or more.
